(19) United States
(12) Patent Application Publication
(10) Pub. No.: US 2003/0044352 A1
(43) Pub. Date: Mar. 6, 2003

(54) Method for evaluating the efficacy of treatment with bacterial DNA and bacterial cell walls

(76) Inventors: Nigel C. Phillips, Point Claire (CA); Mario C. Filion, Laval (CA)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/127,880

(22) Filed: Apr. 23, 2002

Related U.S. Application Data
(60) Provisional application No. 60/286,127, filed on Apr. 24, 2001.

(51) Int. Cl. A61K 49/00; C12Q 1/68

(52) U.S. Cl. 424/9.2; 435/6

(57) ABSTRACT

This invention provides a method for evaluating efficacy of treatment of animals and humans using compositions comprising bacterial DNA and bacterial DNA preserved and complexed on bacterial cell walls. Biological markers such as cytokines, soluble FAS ligand (sFasL) and apoptosis markers released from cells following treatment are measured to assess efficacy of treatment and to identify non-responding patients. This method facilitates identification of animals and humans who should and who should not receive therapeutic administration of these bacterial compositions, and provide a means to evaluate efficacy of such treatment.

### PRIOR RELATED APPLICATIONS

The present application claims priority to U.S. provisional patent application serial number 60/286,127 filed Apr. 24, 2001.

### FIELD OF THE INVENTION

The present invention relates to a method for evaluating an immune response using compositions comprising bacterial DNA and bacterial DNA preserved and complexed on bacterial cell walls. Biological markers such as cytokines, soluble FAS ligand (sFasL) and apoptosis markers released from cells following treatment are measured to assess efficacy of treatment and to identify non-responding patients.

### BACKGROUND OF THE INVENTION

Apoptosis is a genetically programmed, non-inflammatory, energy-dependent form of cell death in tissue including, but not limited to, adult tissue. Aberrations in the regulation of cell proliferation, cell apoptosis, or a combination of the two, are associated with the pathogenesis of a variety of diseases including, but not limited to, cancer, neurodegeneration, autoimmunity, heart disease, and viral infections. Dysfunction of the immune system has also been linked to the development of autoimmune diseases and cancer.

Apoptosis can be initiated by ligands which bind to cell surface receptors including, but not limited to, Fas (French et al., J. Cell Biology, 133:355-64, 1996) and tumor necrosis factor 1 (TNFR1). FasL binding to Fas may initiate intracellular signaling resulting in the activation of cysteine aspartyl proteases (capsases) that initiate the lethal proteolytic cascade of apoptosis execution, which is associated with nuclear DNA-fragmentation, release of nuclear matrix proteins (NuMA) and loss of cell substrate contact (Muzio et al., Cell, 85:817-27, 1996).

Various biological molecules, such as cytokines, FasL, and NuMA, are associated with cells of the immune system or with cells undergoing cell death and apoptosis. Cytokines, such as interleukin-12 (IL-12) and IL-18, are important mediators of immune responses.

IL-12 is an inducible cytokine synthesized predominately by B lymphocytes, dendritic cells and macrophages. IL-12, alone and in combination with other cytokines, promotes the maturation of leukocytes including, but not limited to, B-lymphocytes, CD4+cells, CD8+cells, and NK cells, and induces the secretion of interferon-gamma. IL-12 has been shown to possess potent anti-tumor activity following systemic or local administration in human and mice bearing a variety of malignancies (Brunda et al., J. Exp. Med., 178:1223, 1993; Rook et al., Blood, 94:902, 1999). IL-12 was the first cytokine shown to be capable of eradicating established tumors. IL-12 appears to exert its anti-tumor activity by stimulating natural killer (NK) cells and T lymphocytes, enhancing cytokine expression, and blocking angiogenesis (Stern et al., Life Sci. 58:639, 1996). IL-12 has been shown to possess anti-tumor activity against murine bladder cancer, which indicates a clinical application of IL-12 against human bladder cancer (Eto et al., J. Urol. 163:1549, 2000). The presence of high levels of IL-12 is a favorable prognostic indicator for patients with solid tumors, either as a baseline condition or in response to immunotherapy (Lissoni et al., J. Biol. Regul. Homeost. Agents, 12:38, 1998).

IL-18 is produced predominately by monocytes, macrophages and epidermal cells. IL-1 8 also exerts its activity by stimulating NK cells, T lymphocytes, enhancing cytokine expression and blocking angiogenesis (Cao et al., FASEB J., 13:2195, 1999; Dinarello, Methods, 19:121, 1999). IL-18 displays anti-tumor activity in vivo (Micaleff et al., Cancer Immunol. Immunother., 43:361, 1997). Although the function of IL-18 appears to be identical to that reported for IL-12, the mechanism of activation is different (Dinarello, Methods, 19:121, 1999). For example, it has been shown that IL-18 can augment IFN-gamma synthesis, proliferation and IL-2 receptor alpha-chain expression of T helper 1 clone even in the presence of saturating amounts of IL-12. IL-12 and IL-18 have been reported to have synergistic anti-tumor effects in a mouse bladder cancer model (Yamanaka et al., Cancer Immunol. Immunother., 48:297, 1999).

Fas ligand (FasL) is a type II membrane receptor predominately expressed in activated T lymphocytes and NK cells. Fas, the receptor for FasL, is a type I membrane receptor expressed in various tissues. Binding of FasL to Fas receptors on target tissue cells triggers a cascade of events that ends in apoptosis (Nagata, Ann. Rev. Genetics, 33:29, 1999). Fas/FasL mediates the killing of cancer cells by cytotoxic T lymphocytes and NK cells.

Cytotoxic T-lymphocytes also express Fas but are protected from their own FasL. Some cancer cells express their own FasL. Consequently, a “Fas counterattack” mechanism has emerged, whereby cancer cell FasL will bind to anti-tumor T cell Fas and delete anti-tumor T cells by Fas-mediated apoptosis. FasL may be up-regulated in various cancer cells leading to the elimination of tumor-infiltrating lymphocytes by apoptosis (O'Connell et al., J. Immunol., 160:5669, 1998).

FasL can also be cleaved from the cell surface by metalloproteinases, releasing an active soluble form of the molecule termed soluble Fas ligand (sFasL) (Kayagaki et al., J. Exp. Med., 182:1777, 1995). Although sFasL can induce apoptosis of highly sensitive cancer cells, sFasL is different from the membrane-associated FasL. Whereas FasL has been shown to kill both immune and cancer cells by apoptosis, sFasL has been found to protect resting and activated T lymphocytes from killing by FasL (Suda et al., J. Exp. Med., 186:2045, 1997). Furthermore, sFasL acts as a chemotactic molecule for human neutrophils (Ottonello et al., J. Immunol., 162:3601, 1999).

NuMA, a nuclear mitotic apparatus protein, is released from cells undergoing apoptosis (Miller et al., Biotechniques, 15:1042, 1993). NuMA has been detected in the serum of patients with a wide range of cancers (Miller et al., Cancer Res., 52:422, 1992), and specifically in the urine of patients with bladder cancer (Stampfer et al., J. Urol., 159:394, 1998).

Effective disease treatment with therapeutic compositions is a goal of modem medicine. Most anticancer agents are designed to either kill cancer cells directly (induction of apoptosis) or to stimulate immune response of patients. Often it is difficult to determine whether an animal or human is responding to therapeutic intervention until the therapy has been administered for an extended period of time. Chemotherapy and other forms of therapy such as radiation frequently have deleterious and toxic effects on the recipient and cause numerous other problems. A method for quickly determining whether a specific therapy is effective would be useful for health care professionals such as physicians and veterinarians so that the therapy may be discontinued if it is ineffective, thereby minimizing deleterious and toxic effects on the recipient. What is needed are measures of the efficacy of therapeutic intervention.

The rate of apoptosis increases in tumors responding to irradiation, cytotoxic chemotherapy, heating and hormone ablation. Tumor progression and tumor therapy, such as irradiation or chemotherapeutic treatment, alter various functions of the immune system. M-DNA and MCC has been shown to exert anticancer activity by both directly inducing apoptosis of cancer cells (chemotherapeutic-like activity) and by stimulating the immune system (immunotherapeutic activity) (Filion and Phillips Exp Opin Investig Drugs 2001; 10:2157-2165). What is needed are methods to determine the efficacy of treatment of disease using these and other therapeutic compositions. Therefore, measuring biological markers associated with apoptosis or dysfunction of the immune system could provide a means of measuring the efficacy of cancer therapy. Similarly, these biological markers may be measured to monitor human or animal response to other types of therapy.

### SUMMARY OF THE INVENTION

The present invention provides a method to evaluate the response of animals and humans to administration of immune stimulatory and/or apoptosis-inducing compositions comprising bacterial DNA (B-DNA) administered with a pharmaceutically acceptable carrier, or B-DNA complexed to bacterial cell walls (BCC) and administered with a pharmaceutically acceptable carrier. The response of the animal or the human is measured following administration of the composition. Such responses are demonstrated by cells of the immune system and by cells undergoing apoptosis and by other cells.

The evaluation of the response in the animal or human involves measuring the levels of one or more biological molecules in a biological fluid, cell extract or tissue extract. Such biological molecules include, but are not limited to, cytokines and other indicators of immune function, and apoptosis related markers. These biological markers include without limitation, interleukins (IL) such as IL-12 and IL-18, the protein soluble Fas ligand (sFasL) and nuclear mitotic apparatus protein (NuMA). An change in one or more biological molecules derived from the animal or human receiving the compositions indicates a response to the compositions. Changes may be an increase or decrease in the level of a specific biological molecule when compared to the level prior to administration of the composition. A decrease in the level of a biological molecule such as a molecule which promotes disease progression may indicate a therapeutic effect. An increase in the level of a biological molecule, such as a molecule which assists the immune system infighting disease may indicate a therapeutic effect. A lack of response to the composition suggests that the animal or human receiving the composition may not respond to the composition and is not a candidate for continued administration of the composition. In another embodiment, cells may be obtained from the animal or human and tested in vitro by exposing them to B-DNA, BCC or other bacterial compositions of the invention. The response may be evaluated by measuring the difference in the levels of one or more biological molecules in culture medium, or within the cells before and after administration of the composition. In this manner, an animal's or human's cells, for example tumor cells or cells of the immune system, may be evaluated for responsiveness in vitro before a decision is made to administer the compositions in vivo.

Biological fluids, cell extracts and tissue extracts from animals and humans include but are not limited to urine, blood, serum, plasma, semen, spinal fluid, peritoneal fluid, saliva, sputum, breast exudate, prostatic fluid, extracts of various tissues including the prostate, bladder, ovary, breast, testes, lymph nodes, and tumors. Any biological fluid, cell extract or tissue extract may be evaluated for responsiveness to administration of the bacterial compositions of the present invention.

Through the method of the present invention, animals and humans who respond to treatment with the compositions of the present invention are identified. Further, animals and humans who do not respond to treatment with the compositions of the present invention are also identified, thereby providing a basis for termination of the therapy and possible introduction of an alternative therapy in these individuals. The compositions of the present invention may be administered to animals or humans with or without disease in order to assess the responsiveness of the animal or the human to the composition. In a preferred embodiment, the animal or human receiving the composition has a disease. Such diseases include but are not limited to cancer, autoimmune disease and inflammatory disease. In preferred embodiments, the disease is cancer or a disease of the immune system, such as an autoimmune disease. Types of cancer include but are not limited to bladder, leukemia, prostate, renal, uterine, ovarian, breast, colon, cervical and lung cancer. Types of autoimmune disease include but are not limited to interstitial cystitis, multiple sclerosis, rheumatoid arthritis, diabetes mellitus type I, autoimmune thrombocytopenia purpura, myasthenia gravis, psoriasis vulgaris and systemic lupus erythematosus.

The immune stimulatory and/or apoptosis-inducing compositions of the present invention comprise bacterial DNA (B-DNA) and a pharmaceutically acceptable carrier, or B-DNA complexed to bacterial cell walls (BCC) and combined with a pharmaceutically acceptable carrier. As used herein, “B-DNA” means bacterial DNA and “BCC” means bacterial cell wall complex, whereby B-DNA is complexed on a bacterial cell wall. The compositions of the present invention and methods of making them have been described in PCT/CA/98/00744 and PCT/CA00/00342, which are incorporated herein by reference in their entirety.

The B-DNA and bacterial cell walls may be derived from Mycobacterium species, Bordatella species, Rhodococcus species, Corynebacterium species, Nocardia species, Wisteria species, or Escherichia species. In a preferred embodiment of the present invention, the B-DNA is derived from Mycobacterium species and the bacterial cell walls are derived from Mycobacterium species. Mycobacterium species include, but are not limited to, M. smegmatis, M. fortuitous, M. kansaii, M. tuberculosis, M. bovis, M. avium, and M. phlei. A preferred embodiment of the present invention includes administration of a composition comprising M. phlei-DNA (M-DNA) and a pharmaceutically acceptable carrier, and evaluation of the response. Another preferred embodiment of the present invention includes administration of a composition comprising M-DNA preserved and complexed to M. phlei cell walls (MCC) and a pharmaceutically acceptable carrier, and evaluation of the response. As used herein, “M-DNA” means M. phlei DNA and MCC means M. phlei cell wall complex, whereby M-DNA is complexed on M. phlei cell walls. These compositions promote immune responses including, but not limited to, inhibition of proliferation of and induction of apoptosis in responsive cells including, but not limited to cancer cells, and stimulation of responsive cells of the immune system to produce bioactive molecules. Additional responses include decreases in levels of pathogenic molecules that promote disease progression.

The B-DNA compositions and BCC compositions of the present invention may be administered to an animal or a human through several routes commonly known to one of ordinary skill in the art. Methods of in vivo administration of these compositions, or of formulations comprising such compositions and other materials such as carriers of the present invention that are particularly suitable for various forms include, but are not limited to the following types of administration, oral (e.g. buccal or sublingual), anal, rectal, as a suppository, topical, parenteral, aerosol, inhalation, intravenous, intraarterial, intrathecal, intraperitoneal, transdermal, intradennal, subdeimal, intramuscular, intrauterine, intravesical, intraprostatic, intraurethral, vaginal, into a body cavity, surgical administration at the location of a tumor or internal injury, directly into tumors, into the lumen or parenchyma of an organ, and into bone marrow. However, in some circumstances, BCC and MCC are preferably not administered intravenously or intraarterially.

Accordingly, it is an object of the present invention to provide a method to identify animals or humans who respond or do not respond to administration of compositions comprising B-DNA or BCC and a pharmaceutically acceptable carrier.

It is another object of the present invention to provide a method to identify animals or humans with a disease who respond or do not respond to administration of compositions comprising B-DNA or BCC and a pharmaceutically acceptable carrier.

Yet another object of the present invention is to provide a method to analyze the efficacy of administration of compositions comprising B-DNA or BCC and a pharmaceutically acceptable carrier to animals or humans with a disease.

Still another object of the present invention is to provide a method to analyze the efficacy of administration of compositions comprising B-DNA or BCC and a pharmaceutically acceptable carrier to animals or humans with a disease, wherein the disease is cancer, inflammatory disease or a disease of the immune system.

Another object of the present invention is to provide a method to identify animals or humans with cancer who respond or do not respond to administration of compositions comprising B-DNA or BCC and a pharmaceutically acceptable carrier.

Another object of the present invention is to provide a method to identify animals or humans with a disease of the immune system who respond or do not respond to administration of compositions comprising B-DNA or BCC and a pharmaceutically acceptable carrier.

Yet another object of the present invention is to provide a method to identify animals or humans with inflammatory disease who respond or do not respond to administration of compositions comprising B-DNA or BCC and a pharmaceutically acceptable carrier.

Accordingly, it is an object of the present invention to assess the response of animals or humans to administration of compositions comprising B-DNA or BCC and a pharmaceutically acceptable carrier by measuring levels of biological molecules in biological fluids, cellular extracts or tissue extracts.

It is another object of the present invention to assess the response of animals or humans to administration of compositions comprising B-DNA or BCC and a pharmaceutically acceptable carrier by measuring levels of biological molecules in biological fluids, cellular extracts or tissue extracts, wherein the biological molecules are cytokines, other indicators of immune function, apoptosis related markers or combinations thereof.

It is a specific object of the present invention to assess the response of animals or humans to administration of compositions comprising B-DNA or BCC and a pharmaceutically acceptable carrier by measuring levels of biological molecules in biological fluids, cellular extracts or tissue extracts, wherein the biological molecules are IL-12, IL-18, the protein soluble Fas ligand (sFasL) and nuclear mitotic apparatus protein (NuMA), or combinations thereof.

Another object of the present invention is to provide a method to identify animals or humans with cancer who respond or do not respond to administration of compositions comprising B-DNA or BCC and a pharmaceutically acceptable carrier, wherein the cancer is bladder, leukemia, prostate, renal, uterine, ovarian, breast, colon, cervical or lung cancer.

Another object of the present invention is to provide a method to identify animals or humans with a disease of the immune system who respond or do not respond to administration of compositions comprising B-DNA or BCC and a pharmaceutically acceptable carrier, wherein the disease of the immune system is interstitial cystitis, multiple sclerosis, rheumatoid arthritis, diabetes mellitus type I, autoimmune thrombocytopenia purpura, myasthenia gravis, psoriasis vulgaris or systemic lupus erythematosus.

These and other objects, features and advantages of the present invention will become apparent after a review of the following detailed description of the disclosed embodiments and the appended claims.

### DETAILED DESCRIPTION OF THE INVENTION

The present invention may be understood more readily by reference to the following detailed description of specific embodiments included herein.

The present invention provides a method to evaluate a biological response of an animal or a human to administration of B-DNA or BCC or related compositions. Such administration may occur in vitro or in vivo and may occur in healthy recipients or in recipients with disease. The method of the present invention enables identification of animals and humans who respond or do not respond to administration of the compositions of the present invention by measuring the levels of biological molecules before and after administration of these compositions. This method provides useful information to health care providers concerning the suitability of individuals for receiving the therapeutic compositions of the present invention. This method further enables determination of the efficacy of the therapeutic compositions of the present invention. This method also provides a basis for terminating administration of these therapeutic compositions if they are ineffective.

The immune stimulatory and/or apoptosis-inducing compositions of the present invention comprise bacterial DNA (B-DNA) and a pharmaceutically acceptable carrier, or B-DNA complexed to bacterial cell walls (BCC) and combined with a pharmaceutically acceptable carrier. As used herein, “B-DNA” means bacterial DNA and “BCC” means bacterial cell wall complex, whereby B-DNA is complexed on a bacterial cell wall. The compositions of the present invention and methods of making them have been described in PCT/CA/98/00744 and PCT/CA00/00342, which are incorporated herein by reference in their entirety.

The B-DNA and bacterial cell walls may be derived from Mycobacterium species, Bordatella species, Rhodococcus species, Corynebacterium species, Nocardia species, Listeria species, or Escherichia species. In a preferred embodiment of the present invention, the B-DNA is derived from Mycobacterium species and the bacterial cell walls are derived from Mycobacterium species. Mycobacterium species include, but are not limited to, M. smegmatis, M. fortuitous, M. kansaii, M. tuberculosis, M. bovis, M. avium, and M. phlei. A preferred embodiment of the present invention includes administration of a composition comprising M. phlei-DNA (M-DNA) and a pharmaceutically acceptable carrier, and evaluation of the response. Another preferred embodiment of the present invention includes administration of a composition comprising M-DNA preserved and complexed to M phlei cell walls (MCC) and a pharmaceutically acceptable carrier, and evaluation of the response. As used herein, “M-DNA” means M. phlei DNA and MCC means M. phlei cell wall complex, whereby M-DNA is complexed on M. phlei cell walls. These compositions promote immune responses including, but not limited to, inhibition of proliferation of and induction of apoptosis in responsive cells including, but not limited to cancer cells, and stimulation of responsive cells of the immune system to produce bioactive molecules. Additional responses include decreases in levels of pathogenic molecules that promote disease progression.

Pharmaceutically Acceptable Carriers

The terms “pharmaceutically acceptable carrier” or “pharmaceutically acceptable vehicle” are used herein to mean, without limitation, any liquid, solid or semi-solid, including, but not limited to, water or saline, a gel, cream, salve, solvent, diluent, fluid ointment base, ointment, paste, implant, liposome, micelle, giant micelle, and the like, which is suitable for use in contact with living animal or human tissue without causing adverse physiological responses, and which does not interact with the other components of the composition in a deleterious manner. Other pharmaceutically acceptable carriers or vehicles known to one of skill in the art may be employed to make compositions for delivering the bacterial DNA and cell wall compositions of the present invention.

The bacterial DNA and cell wall compositions of the present invention may be combined with pharmaceutically acceptable carriers and administered as compositions in vitro or in vivo. Forms of administration include, but are not limited to, injections, solutions, creams, gels, implants, pumps, ointments, emulsions, suspensions, microspheres, particles, microparticles, nanoparticles, liposomes, pastes, patches, tablets, transdermal delivery devices, sprays, aerosols, or other means familiar to one of ordinary skill in the art. Such pharmaceutically acceptable carriers are commonly known to one of ordinary skill in the art. Pharmaceutical formulations of the present invention can be prepared by procedures known in the art using well known and readily available ingredients. For example, the compounds can be formulated with common excipients, diluents, or carriers, and formed into tablets, capsules, suspensions, powders, and the like. Examples of excipients, diluents, and carriers that are suitable for such formulations include the following: fillers and extenders (e.g., starch, sugars, mannitol, and silicic derivatives); binding agents (e.g., carboxymethyl cellulose and other cellulose derivatives, alginates, gelatin, and polyvinyl-pyrrolidone); moisturizing agents (e.g., glycerol); disintegrating agents (e.g., calcium carbonate and sodium bicarbonate); agents for retarding dissolution (e.g., paraffin); resorption accelerators (e.g., quaternary ammonium compounds); surface active agents (e.g., cetyl alcohol, glycerol monostearate); adsorptive carriers (e.g., kaolin and bentonite); emulsifiers; preservatives; sweeteners; stabilizers; coloring agents; perfuming agents; flavoring agents; lubricants (e.g., talc, calcium and magnesium stearate); solid polyethyl glycols; and mixtures thereof.

The formulations can be so constituted that they release the active ingredient only or preferably in a particular location, possibly over a period of time. Such combinations provide yet a further mechanism for controlling release kinetics. The coatings, envelopes, and protective matrices may be made, for example, from polymeric substances or waxes.

Compositions comprising B-DNA or BCC and a pharmaceutically acceptable carrier are prepared by uniformly and intimately bringing into association the sequence and the pharmaceutically acceptable carrier. Pharmaceutically acceptable carriers include liquid carriers, solid carriers or both. Liquid carriers are aqueous carriers, non-aqueous carriers or both, and include, but are not limited to, aqueous suspensions, oil emulsions, water-in-oil emulsions, water-in-oil-in-water emulsions, site-specific emulsions, long-residence emulsions, sticky-emulsions, microemulsions and nanoemulsions. Solid carriers are biological carriers, chemical carriers or both and include, but are not limited to, viral vector systems, particles, microparticles, nanoparticles, microspheres, nanospheres, minipumps, bacterial cell wall extracts and biodegradable or non-biodegradable natural or synthetic polymers that allow for sustained release of the bacterial compositions. Emulsions, minipumps and polymers can be implanted in the vicinity of where delivery is required (Brem et al. J. Neurosurg. 74: 441, 1991). Methods used to complex B-DNA or BCC to a solid carrier include, but are not limited to, direct adsorption to the surface of the solid carrier, covalent coupling to the surface of the solid carrier, either directly or via a linking moiety, and covalent coupling to the polymer used to make the solid carrier. Optionally, B-DNA or BCC can be stabilized by the addition of non-ionic or ionic polymers such as polyoxyethylenesorbitan monooleates (TWEENs) or hyaluronic acid.

Preferred aqueous carriers include, but are not limited to, water, saline and pharmaceutically acceptable buffers. Preferred non-aqueous carriers include, but are not limited to, a mineral oil or a neutral oil including, but not limited to, a diglyceride, a triglyceride, a phospholipid, a lipid, an oil and mixtures thereof, wherein the oil contains an appropriate mix of polyunsaturated and saturated fatty acids. Examples include, but are not limited to, soybean oil, canola oil, palm oil, olive oil and myglyol, wherein the fatty acids can be saturated or unsaturated. Optionally, excipients may be included regardless of the pharmaceutically acceptable carrier used to present the B-DNA or BCC compositions to cells. These excipients include, but are not limited to, anti-oxidants, buffers, and bacteriostats, and may include suspending agents and thickening agents.

Methods of in vivo administration of the compositions of the present invention, or of formulations comprising such compositions and other materials such as carriers of the present invention that are particularly suitable for various forms include, but are not limited to the following types of administration, oral (e.g. buccal or sublingual), anal, rectal, as a suppository, topical, parenteral, aerosol, intravenous, intraarterial, inhalation, intrathecal, intraperitoneal, transdermal, intradermal, subdermal, intramuscular, intrauterine, intravesical, intraprostatic, intraurethral, vaginal, into a body cavity, surgical administration at the location of a tumor or internal injury, directly into tumors, into the lumen or parenchyma of an organ, and into bone marrow. However, in some cases, BCC and MCC are preferably not delivered intravenously or intraarterially. Techniques useful in the various forms of administrations mentioned above include but are not limited to, topical application, ingestion, surgical administration, injections, sprays, transdermal delivery devices, osmotic pumps, electrodepositing directly on a desired site, or other means familiar to one of ordinary skill in the art. Sites of application can be external, such as on the epidermis, or internal, for example a gastric ulcer, a surgical field, or elsewhere.

The compositions of the present invention can be applied in the form of creams, gels, solutions, suspensions, liposomes, particles, or other means known to one of skill in the art of formulation and delivery of the compositions. Ultrafine particle sizes can be used for inhalation delivery of therapeutics. Some examples of appropriate formulations for subcutaneous administration include but are not limited to implants, depot, needles, capsules, and osmotic pumps. Some examples of appropriate formulations for vaginal administration include but are not limited to creams and rings. Some examples of appropriate formulations for oral administration include but are not limited to: pills, liquids, syrups, and suspensions. Some examples of appropriate formulations for transdermal administration include but are not limited to gels, creams, pastes, patches, sprays, and gels. Some examples of appropriate delivery mechanisms for subcutaneous administration include but are not limited to implants, depots, needles, capsules, and osmotic pumps. Formulations suitable for parenteral administration include but are not limited to aqueous and non-aqueous sterile injection solutions which may contain anti-oxidants, buffers, bacteriostats and solutes which render the formulation isotonic with the blood of the intended recipient, and aqueous and non-aqueous sterile suspensions which may include suspending agents and thickening agents. Extemporaneous injection solutions and suspensions may be prepared from sterile powders, granules and tablets commonly used by one of ordinary skill in the art.

Embodiments in which the compositions of the invention are combined with, for example, one or more pharmaceutically acceptable carriers or excipients may conveniently be presented in unit dosage form and may be prepared by conventional pharmaceutical techniques. Such techniques include the step of bringing into association the compositions containing the active ingredient and the pharmaceutical carrier(s) or excipient(s). In general, the formulations are prepared by uniformly and intimately bringing into association the active ingredient with liquid carriers. Preferred unit dosage formulations are those containing a dose or unit, or an appropriate fraction thereof, of the administered ingredient. It should be understood that in addition to the ingredients particularly mentioned above, formulations comprising the compositions of the present invention may include other agents commonly used by one of ordinary skill in the art.

The volume of administration will vary depending on the route of administration. Such volumes are known to one of ordinary skill in the art of administering compositions to animals or humans. Depending on the route of administration, the volume per dose is preferably about 0.001 to 100 ml per dose, more preferably about 0.01 to 50 ml per dose and most preferably about 0.1 to 30 ml per dose. For example, intramuscular injections may range in volume from about 0.1 ml to 1.0 ml. The compositions administered alone, or together with other therapeutic agent(s), can be administered in a single dose treatment, in multiple dose treatments, or continuously infused on a schedule and over a period of time appropriate to the disease being treated, the condition of the recipient and the route of administration. Moreover, the other therapeutic agent can be administered before, at the same time as, or after administration of the compositions.

Preferably, the amount of B-DNA or BCC composition administered per dose is from about 0.0001 to 100 mg/kg, more preferably from about 0.001 to 10 mg/kg and most preferably from about 0.01 to 5 mg/kg. In one preferred embodiment, the B-DNA or BCC compositions, in combination with a chemotherapeutic agent, is administered to an animal or human in an amount effective to evaluate whether the B-DNA or BCC compositions add to, antagonize, synergize with or potentiate the anti-neoplastic effect of the chemotherapeutic agent. Preferably, the amount of therapeutic agent administered per dose is from about 0.001 to 1000 mg/kg, more preferably from about 0.01 to 500 mg/kg and most preferably from about 0.1 to 100 mg/kg. The particular sequence and the particular therapeutic agent administered, the amount per dose, the dose schedule and the route of administration should be decided by the practitioner using methods known to those skilled in the art and will depend on the type of disease, the severity of the disease, the location of the disease and other clinical factors such as the size, weight and physical condition of the recipient. In addition, in vitro assays may optionally be employed to help identify optimal ranges for B-DNA or BCC and for B-DNA or BCC plus therapeutic agent administration. These assays may employ various cells cultured in vitro, such as tumor cells or cells of the immune system.

Compositions according to this invention will promote immune responses including, but not limited to, inhibition of proliferation of and induction of apoptosis in responsive cells including, but not limited to cancer cells, and stimulation of responsive cells of the immune system to produce bioactive molecules.

B-DNA and BCC compositions according to this invention may be administered to a healthy human or animal or an animal or human with disease, for example cancer, autoimimune disease, inflammatory disorders, or other diseases associated with aberrations in apoptosis or dysfunction in immune response. Cancers include, but are not limited to, squamous cell carcinoma, fibrosarcoma, sarcoid carcinoma, melanoma, mammary cancer, lung cancer, colorectal cancer, renal cancer, osteosarcoma, cutaneous melanoma, basal cell carcinoma, pancreatic cancer, bladder cancer, brain cancer, ovarian cancer, uterine cancer, prostate cancer, leukemia, lymphoma and metastases derived therefrom. Autoimmune diseases include, but are not limited to, interstitial cystitis, multiple sclerosis, rheumatoid arthritis, diabetes mellitus type I, autoimmune thrombocytopenia purpura, myasthenia gravis, psoriasis vulgaris and systemic lupus erythematosus. Inflammatory disorders include, but are not limited to, interstitial cystitis.

Compositions according to this invention may be administered in a suitable dosage range determined by factors including, but not limited to, the type of composition being administered, the route of administration, the disease being treated, and the weight and physical condition of the human or animal being treated. For example, when treating bladder cancer, M-DNA complexed on MCC may be administered intravesically in a range of about 1 to about 16 mg per dose, more preferably from about 2 to about 12 mg per dose, and most preferably from about 4 to about 8 mg per dose. When treating prostate cancer, M-DNA complexed on MCC may be administered intraprostatically in a range of about 1 to about 5000 μg per dose, more preferably from about 10 to about 2000 μg per dose, and most preferably from about 100 to about 1000 μg per dose. The schedule of administering dosages of compositions according to this invention may be determined by a practitioner using methods know to those skilled in the art.

After administering compositions according to this invention, biological fluids, cell samples or tissue samples may be collected to obtain biological markers for measurement. The body fluids may include, but are not limited to, urine, blood, serum, plasma, semen, spinal fluid, peritoneal fluid, saliva, sputum and breast exudates. A practitioner may choose the body fluid or tissue to be collected based on the type of disease being treated. In another embodiment, cells may be obtained from the animal or human and tested in vitro by exposing them to B-DNA, BCC or other bacterial compositions of the invention. Methods of culturing cells in vitro, harvesting culture medium and homogenizing cells for determination of intracellular levels of biological molecules are known to those of ordinary skill in the art. The response may be evaluated by measuring the difference in the levels of one or more biological molecules in culture medium, or within the cells, before and after administration of the composition. In this manner, an animal's or human's cells, for example tumor cells or cells of the immune system, may be evaluated for responsiveness in vitro before a decision is made to administer the compositions in vivo. Cell culture fluids may be used for cells or tissues cultured in vitro.

Depending on the route of administration of the B-DNA or BCC compositions, the collection time of the biological fluid is preferably about 3 to 24 hours post-treatment, more preferably about 6 to 18 hours post-treatment and most preferably about 6 to 8 hours post-treatment. Alternatively, the biological fluids or tissue samples may be collected at appropriate times determined by the methods to be used to detect the biological markers. For example, collection times for IL-12, IL-18, sFasL, and NuMA may be 6 to 8 hours depending on whether the biological markers will be detected using RT-PCR or ELISA. It is to be understood that biological fluids, cell samples or tissue samples may be obtained prior to administration of the compositions of the present invention to facilitate comparison of the levels of the biological molecules before and after administration of the compositions. However, in some circumstances, responding and non-responding animals and humans may be identified based on levels of biological markers following administration of the bacterial compositions of the invention in comparison to clinical values observed for healthy animals or humans or for animals or humans with specific diseases.

After the body fluids or tissue samples are collected, biological molecules or markers are measured by methods know to those skilled in the art including, but not limited to, ELISA, flow cytometry, ELISPOT, RT-PCR, biological assays and in situ hybridization. Biological markers may include, but are not limited to, IL-12, IL-18, sFasL, and NuMA. If the biological markers will not be measured immediately, body fluids, cells or tissue samples containing the markers may be refrigerated or frozen because cytokines, such as IL-12 and IL-18, are unstable at room temperature. Body fluids may be kept in a sterile container at 4° C. for 24 hours. More preferably, body fluids, cells or tissue samples may be stored at −20° C. for 3-6 months. Most preferably, body fluids, cells or tissue samples may be stored at −80° C. for more than 6 months.

Following determination of the levels of the selected biological molecule before and after in vitro or in vivo administration of the bacterial compositions of the invention, the changes, or lack thereof, in the levels of a biological molecule are determined. No change indicates a non-responding animal or human. Non-responding animals or humans are not considered as good candidates for continued application of the therapeutic bacterial compositions of the invention. As stated above, responding animals and humans may show changes in the level of the biological molecule which may be evidenced as an increase or a decrease. Such an increase or a decrease is to be interpreted in view of the specific molecule being measured. A decrease in a pathogenic molecule may be interpreted as a favorable response. An increase in a specific disease-fighting interleukin may also be interpreted as a favorable response. An increase in biological molecules associated with apoptosis of cancer cells may also be interpreted as a favorable response. An augmentation by the bacterial compositions of the present invention of a disease-fighting interleukin stimulated by another therapy may also be interpreted as a favorable response. Animals and humans demonstrating favorable responses are considered as good candidates for continued application of the therapeutic bacterial compositions of the invention. Alternatively, an increase in a pathogenic molecule may be interpreted as an unfavorable response, even though the recipient responded. Unfavorable responses in animals or humans would suggest that they are not considered as good candidates for continued application of the therapeutic bacterial compositions of the invention. Alternatively, when levels of the biological molecule are not measured both before and after administration of the bacterial compositions of the invention, comparison to post-administration levels may be performed with reference to clinical values appropriate for the clinical status of the recipient. For example, the recipient may be healthy, may have interstitial cystitis or prostate cancer.

The present invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention.

### EXAMPLE 1

### Clinical Study on Bladder Cancer

Fourteen patients with carcinoma in situ (CIS) of the bladder who failed to respond to BCG treatment were enrolled in 9 centers in Australia and Canada (see Table 1).

TABLE 1

| Patient | Response* at week 12 |
|---|---|
| 001 | CR |
| 002 | CR |
| 003 | PR |
| 004 | PR |
| 005 | CR |
| 006 | PR |
| 007 | PR |
| 008 | PR |
| 009 | CR |
| 010 | PR |
| 011 | F |
| 012 | F |
| 013 | F |
| 014 | F |
| 015 | F |
| 016 | F |
| 017 | F |

*CR is defined as a complete response, determined by negative biopsy and negative urine cytology at week 12, where negative means no detectable cancer cells. PR is defined as a partial response, determined by negative biopsy and by positive urine cytology at week 12. F is defined as a failure, determined by positive biopsy and positive urine cytology at week 12.

### Mycobacterial Cell Wall-DNA Complex Treatment

Each CIS patient was treated once weekly for a period of 6 weeks with 4 mg of emulsified mycobacterial cell wall-DNA complex (MCC) in a volume of 50 ml of saline administrated intravesically. The emulsified mycobacterial cell wall-DNA complex (MCC) was prepared as described in the patent PCT/CA/98/00744 by using 2% (v/v) of sterile and DNase free mineral oil and 0.2% (v/v) of sterile and DNase free Tween-80. Clinical response to emusified mycobacterial cell wall-DNA complex (CC) treatment was evaluated by biopsy and urine cytology at week 12. (see results in Table 1).

### Urine Collection

Urine was collected before treatment, and at 6 to 24 hours thereafter at 3 and 6 weeks of treatment. Samples were immediately frozen to −20° C. and stored at this temperature until analysis. For analysis, the samples were thawed to a maximum of 4° C. and centrifuged to remove cells and debris. Analyses were then carried out on the supernatant fluid.

### Cytokines, sFASL, NuMA and Creatinine Determination

IL-6, IL-8, IL-12, IL-1 8, NuMA and sFasL in urine were determined using commercially available ELISA kits (IL-6, IL-8 and IL-12 ELISA kits were obtained from BioSource, Camarillo, Calif.; IL-1 8 ELISA kit from R & D Systems, Minneapolis, Minn.; and NuMA and sFasL ELISA kits from Oncogen-Calbiochem, Cambridge, Mass.). Data were standardized to urinary creatinine, which is known to be significantly correlated with the volume of produced urine (p<0.001; de Reijke et al., J. Urol., 155:477, 1996). The level of creatinine present in urine was measured by means of a commercial kit based on the Jaffe reaction (Sigma-Diagnostics, Diagnostics, St-Louis, Mo.).

### Correlation Between the Synthesis of IL-12, IL-18 and sFasL and the efficacy of Mycobacterial Cell Wall-DNA Complex Treatment.

IL-6, IL-8, IL-1 2, IL-1 8, NuMA and sFasL were measured in the urine at 3 and 6 weeks of treatment. Table 2 shows the response of each patient to mycobacterial cell wall-DNA complex (MCC) at week 3 (w3) and 6 (w6). An increase of 50% over the baseline (pre-treatment) level of cytokines, NuMA or sFasL produced was considered to be a positive response.

TABLE 2

| | Re- | | | | | | | | | | | | |
|---|---|---|---|---|---|---|---|---|---|---|---|---|---|
| | sponse | | | | | | | | | | | | |
| Pa- | at | sFASL | | IL-12 | | NuMA | | IL-18 | | IL-6 | | IL-8 | |
| tient | week 12 | w3 | w6 | w3 | w6 | w3 | w6 | w3 | w6 | w3 | w6 | w3 | w6 |
| 001 | CR | + | + | + | + | − | + | − | + | + | + | + | + |
| 002 | CR | + | − | + | + | + | + | + | − | + | + | + | + |
| 003 | PR | − | − | − | + | − | − | − | + | − | + | − | + |
| 004 | PR | − | + | + | + | + | + | − | + | + | + | + | + |
| 005 | CR | + | + | − | + | + | + | + | + | + | + | + | + |
| 006 | PR | + | − | + | + | − | − | − | + | + | + | + | + |
| 007 | PR | − | + | − | + | − | + | − | + | + | + | + | + |
| 008 | PR | − | − | − | + | − | + | − | − | − | + | − | + |
| 009 | CR | − | + | + | + | − | + | + | + | − | − | + | + |
| 010 | PR | − | − | − | − | − | − | + | − | + | − | + | − |
| 011 | F | − | − | − | − | − | − | − | − | − | − | − | + |
| 012 | F | − | nd | − | nd | + | nd | − | nd | + | nd | + | nd |
| 013 | F | nd | − | nd | − | nd | − | nd | nd | nd | + | nd | − |
| 014 | F | − | nd | + | nd | + | nd | nd | nd | + | nd | + | nd |
| 015 | F | − | − | − | − | − | − | − | − | + | + | + | − |
| 016 | F | − | − | − | + | − | − | − | − | + | + | + | + |
| 017 | F | − | − | + | + | + | − | + | + | + | + | + | + |

The numbers of patients producing sFASL, IL-12, NuMA, IL-18, IL-6 and IL-8 in response to mycobacterial cell wall-DNA complex (MCC) at week 3 and 6 were compiled (Table 3).

TABLE 3

| | |
|---|---|
| Week 3 | Week 6 |

| | R* | F** | R | F |
|---|---|---|---|---|
| sFASL | 4/10 | 0/6 | 5/10 | 0/5 |
| IL-12 | 5/10 | 2/6 | 9/10 | 2/5 |
| NuMA | 3/10 | 3/5 | 7/10 | 0/5 |
| IL-18 | 4/10 | 1/5 | 7/10 | 1/4 |
| IL-6 | 7/10 | 5/6 | 8/10 | 4/5 |
| IL-8 | 8/10 | 5/6 | 9/10 | 3/5 |

*R Complete and partial response
**F Failure

Table 3 shows that patients with a positive clinical response to mycobacterial cell wall-DNA complex (MCC) produced sFasL (40% at week 3 and 50% at week 6). No non-responding patients produced sFasL. The incidence of false positives was 0% when sFasL served as a prognostic marker. The use of sFasL as a prognostic marker to identify non-responding patients was significant (p>0.0047; Fisher's exact test). IL-12 was produced by 50 and 90% of responding patients at week 3 and 6, respectively. The incidence of false positives was 33% at weeks 3 and 40% at week 6 when IL-12 served as a prognostic marker. NuMA was produced by 30 and 70% of responding patients at weeks 3 and 6, respectively. The incidence of false positives was 60% at week 3 and 0% at week 6 when NuMA served as a prognostic marker. The use of NuMA as a prognostic marker to identify non-responding patients at week 6 was significant (p>0.0015; Fisher's exact test). IL-18 was produced by 40 and 70% of responding patients at weeks 3 and 6, respectively. The incidence of false positives was 20% at week 3 and 25% at week 6 when IL-18 served as a prognostic marker. IL-6 and IL-8 levels were enhanced following mycobacterial cell wall-DNA complex (MCC) treatment and had no correlation with clinical response. These results demonstrate that sFasL and NuMa are reliable prognostic indicators of clinical efficacy in response to mycobacterial cell wall-DNA complex (MCC) treatment. IL-12 and IL-18 measurements showed that mycobacterial cell wall-DNA complex (MCC) treatment was capable of eliciting the induction of these indicators of anticancer activity, but with a higher false positive response than seen with sFasL and NuMA.

### EXAMPLE 2

### Clinical Studies on Prostate Cancer

Patients with prostate cancer are treated with about 1 μg to about 5000 μg of a nanoparticulate suspension of mycobacterial cell wall-DNA complex (MCC) administrated directly into the tumor by trans-rectal or trans-urethral injection. Urine and blood samples are collected at weekly intervals following treatment. Urine samples are processed as described in Example 1, and blood samples are allowed to clot and serum collected. Measurement of cytokines, sFasL and NuMA demonstrates that elevated levels of sFasL, IL-1 8, IL-12 and NuMA in the urine and serum correlate with enhanced immune response (cellular infiltration) and apoptosis in prostate tumor biopsies and objective reduction in tumor mass, as determined by ultrasound or other imaging techniques.

### EXAMPLE 3

### Clinical Studies on Interstitial Cystitis

Interstitial cystitis is an inflammatory disease of the bladder characterized by suprapubic pain, urinary urgency and frequency of urination. The etiology of interstitial cystitis is still unclear, but possible theories include an autoimmune deficiency associated with a disregulation of the T-helper cell type 1 (Th-1)/T-helper helper cell type 2 (Th-2) balance (Ochs, Clin. Lab. Med. 17:571, 1997; Peters et al., Urology, 54:450, 1999). Patients with interstitial cystitis are treated with nanoparticulate suspension of mycobacterial cell wall-DNA complex administrated intravesically. Urine and blood samples are collected at weekly intervals following treatment. Urine samples are processed as described in Example 1, and blood samples are allowed to clot and serum collected. Measurement of cytokines, sFasL and NuMA demonstrates that elevated levels of sFasL, IL-1 8, IL-12 and NuMA in the urine and serum correlate with the decrease of pain, urinary urgency and frequency of urination.

All patents, publications and abstracts cited above are incorporated herein by reference in their entirety. It should be understood that the foregoing relates only to preferred embodiments of the present invention and that numerous modifications or alterations may be made therein without departing from the spirit and the scope of the present invention as defined in the following claims.

We claim:

1. A method of evaluating a biological response in an animal or human comprising:
administering a composition comprising bacterial DNA (B-DNA) and a pharmaceutically acceptable carrier to the animal or human;
measuring a level of a biological molecule following administration of the composition; and,
determining if the animal or human demonstrated the biological response by comparing the level of the biological molecule to another level of the biological molecule.

2. The method of claim 1, wherein the another level of the biological molecule is derived from clinical values for the biological molecule or from a measurement of the biological molecule performed before administration of the composition.

3. The method of claim 1, further comprising evaluation of the biological response as favorable, absent or unfavorable.

4. The method of claim 3, wherein if the response is unfavorable or absent, subsequent administration of the composition is terminated.

5. The method of claim 3, wherein if the response is favorable, subsequent administration of the composition is continued.

6. The method of claim 1, wherein the biological molecule is a cytokine, an indicator of immune function, an apoptosis related molecule, or a combination thereof.

7. The method of claim 1, wherein the biological molecule is IL-12, IL-18, sFasL, NuMA or a combination thereof.

8. The method of claim 1, wherein the B-DNA is selected from the group consisting of Mycobacterium DNA, Bordatella DNA, Rhodococcus DNA, Corynebacterium DNA, Nocardia DNA, Listeria DNA, and Escherichia DNA.

9. The method of claim 1, wherein the B-DNA is Mycobacterium DNA.

10. The method of claim 1, wherein the B-DNA is Mycobacterium phlei DNA (M-DNA).

11. The method of claim 1, wherein the animal or the human has a disease.

12. The method of claim 11, wherein the disease is cancer, inflammatory disease or an autoimmune disease.

13. A method of evaluating a biological response in an animal or human comprising:
administering a composition comprising bacterial DNA (B-DNA) preserved and complexed on bacterial cell wall (BCC), and a pharmaceutically acceptable carrier, to the animal or human;
measuring a level of a biological molecule following administration of the composition; and,
determining if the animal or human demonstrated the biological response by comparing the level of the biological molecule to another level of the biological molecule.

14. The method of claim 13, wherein the another level of the biological molecule is derived from clinical values for the biological molecule or from a measurement of the biological molecule performed before administration of the composition.

15. The method of claim 13, further comprising evaluation of the biological response as favorable, absent or unfavorable.

16. The method of claim 15, wherein if the response is unfavorable or absent, subsequent administration of the composition is terminated.

17. The method of claim 15, wherein if the response is favorable, subsequent administration of the composition is continued.

18. The method of claim 13, wherein the biological molecule is a cytokine, an indicator of immune function, an apoptosis related molecule, or a combination thereof.

19. The method of claim 13, wherein the biological molecule is IL-12, IL-18, sFasL, NuMA or a combination thereof.

20. The method of claim 13, wherein the bacterial cell wall is selected from the group consisting of Mycobacterium cell wall, Bordatella cell wall, Rhodococcus cell wall, Corynebacterium cell wall, Nocardia cell wall, Listeria cell wall, and Escherichia cell wall.

21. The method of claim 13, wherein the bacterial cell wall is Mycobacterium cell wall.

22. The method of claim 13, wherein the bacterial cell wall is Mycobacterium phlei cell wall (MCC).

23. The method of claim 13, wherein the animal or the human has a disease.

24. The method of claim 23, wherein the disease is cancer, inflammatory disease or an autoimmune disease.

25. A method of evaluating a biological response in cells in vitro from an animal or human comprising:
administering a composition comprising bacterial DNA (B-DNA) and a pharmaceutically acceptable carrier to the cells;
measuring a level of a biological molecule following administration of the composition; and,
determining if the cells demonstrated the biological response by comparing the level of the biological molecule to another level of the biological molecule.

26. The method of claim 25, wherein the another level of the biological molecule is derived from a measurement of the biological molecule performed before administration of the composition.
